(19) United States
(12) Patent Application Publication
(10) Pub. No.: US 2015/0175554 A1
(43) Pub. Date: Jun. 25, 2015

(54) Highly pure bendamustine hydrochloride monohydrate

(71) Applicant: SHILPA MEDICARE LIMITED, Raichur (IN)

(72) Inventors: Vimal Kumar Shrawat, Raichur (IN); Seshachalam Unnam, Raichur (IN); Vinod Kumar Singh, Raichur (IN); Prashant Purohit, Raichur (IN)

(21) Appl. No.: 14/411,399

(22) Filed: Dec. 26, 2014

Related U.S. Application Data
(63) Continuation of application No. 13/635,097, filed on Sep. 14, 2012.

(30) Foreign Application Priority Data
Nov. 1, 2010 (IN) 3261/CHE/2010

(51) Int. Cl. C07D 235/16; C07C 209/10

(52) U.S. Cl. CPC C07D 235/16; C07C 209/10; C07B 2200/13

(57) ABSTRACT

The present invention provide processes for the preparation of highly pure Bendamustine hydrochloride monohydrate of formula (I)

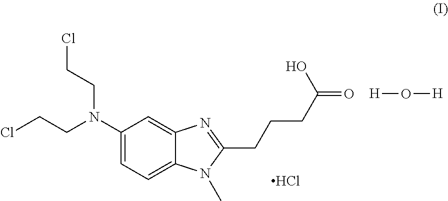

The present application relates to Bendamustine hydrochloride monohydrate crystalline Form-SM characterized by X-ray powder diffraction pattern as depicted in FIG. 1 consisting peaks selected from the XRPD 2 theta degrees peaks at 7.42, 10.60, 11.17, 16.43, 17.94, 22.89, 26.33, 28.77, 30.28, 31.92, and 40.89±0.1 2θ° having a purity of greater than 99.5% (by HPLC).

The present application also provides a process for the preparation of highly pure Bendamustine hydrochloride monohydrate crystalline Form-SM useful in making pharmaceutical composition for the treatment of cancer or similar proliferative disorders.

### RELATED APPLICATIONS

This application is a continuation of U.S. patent application Ser. No. 13/635,097, filed Sep. 14, 2012, now U.S. Patent Publication. No. 2013/0217888, which claims priority under 35 U.S.C. Section 371 to PCT Patent Application No. PCT/IN2011/000585, filed Aug. 29, 2011, which claims priority to IN Patent Application No. 3261/CHE/2010, filed Nov. 1, 2010, the disclosures of each of which are incorporated by reference herein in their entirety.

### FIELD OF THE INVENTION

Particular aspects of the present application relate to the process for preparation Bendamustine hydrochloride monohydrate and its crystalline form designated as “SM”

### BACKGROUND OF THE INVENTION

Bendamustine is a benzimidazole analog and was known since 1963 along with its synthesis (by Ozegowski and Krebs) in the German Democratic Republic (GDR). Initially, it was available from 1971 to 1992 under the Brand name Cytostasan® in GDR. It has been marketed in Germany under the trade name Ribomustin® and widely used for the treatment of Chronic Lymphocytic Leukemia (CLL), Non-Hodgkin's lymphoma (NHL), Multiple myeloma and Breast cancer.

Bendamustine hydrochloride monohydrate is represented by structural formula I.

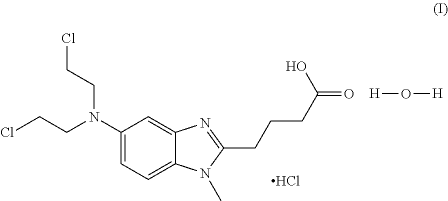

and has chemical name—1H-benzimidazole-2-butanoic acid, 1-methyl-5-[bis(2-chloroethyl)amino]-, monohydrochloride, monohydrate.

In US, Bendamustine hydrochloride is currently sold in the form of a sterile non-pyrogenic white to off-white lyophilized powder in a single use vial, for the treatment of chronic lymphocytic leukemia under the trade name TREANDA®.

Ozegowski et al. has reported for the first time the process for the preparation of bendamustine hydrochloride monohydrate in Zbl. Pharma. 110, (1971) Heft 10, 1013-1019. German Patent DD 34727 also disclosed a process for the preparation of ω-[5-bis(β-chloroethyl)amino-benzimidazolyl-(2)]alkane carboxylic acids substituted in the 1-position.

Zhongguo Xinyao Zazhi (2007), 16(23), 1960-1961, 1970 disclose a process for the preparation of bendamustine hydrochloride monohydrate, which involves reacting 1-methyl-2-(4′-ethyl butyrate)-5-amino]-1H-benzimidazole with ethylene oxide in the presence of water, sodium acetate and acetic acid, by maintaining at 5° C. for 5 hours and overnight at 20° C. to give (4-{5-[bis-(2-hydroxy-ethyl)-amino]-1-methyl-1H-benzimidazol-2-yl}-butyric acid ethyl ester as a jelly mass. This mass on subjecting to chlorination using thionyl chloride in chloroform and subsequent in situ hydrolysis with concentrated HCl at 90-95° C. to provide bendamustine hydrochloride. It also discloses a process for the recrystallization of bendamustine hydrochloride from water and the product obtained is a monohydrate with a melting point of 148-151° C.

Gust et al., in Monatshefte fur Chemie (1997), 128, 291-299 disclose that the known process for preparation of Bendamustine has been performed by an eleven step sequence starting from 2,4-dinitrochlorobenzene, and the crucial conversions are the chlorination of ethyl 4-(6-bis(2-hydroxyethylamino)-3-methylbenzimidazoylbutyrate (di hydroxy ester) with SOCl₂, resulting in ethyl 4-(6-bis(2-chloroethyl)amino-3-methylbenzimidazol-2-ylbutyrate (dichloro ester) and the subsequent ester cleavage with HCl to obtain 4-(6-bis(2-chloroethyl)-amino-3-methylbenzimidazol-2-ylbutyric acid (bendamustine). During these reaction conditions, often bendamustine gets hydrolyzed in trace quantities to form the chlorohydroxy (HP1) and dihydroxy (HP2) derivatives having following structures—

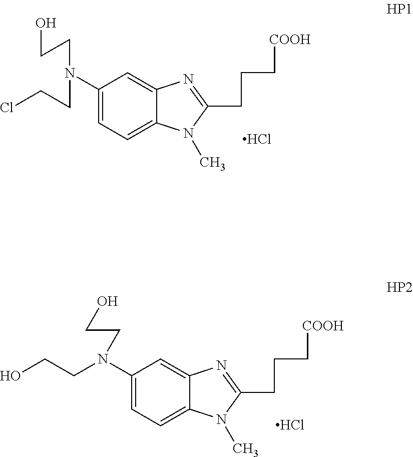 [SMILES: Cn1c(CCCC(=O)O)nc2cc(N(CCO)CCCl)ccc21 ; Cl ; Cn1c(CCCC(=O)O)nc2cc(N(CCO)CCO)ccc21 ; Cl]

In IP.com Journal 2009, 9(7B), 21, a process for the preparation of ethyl-4-[5-[bis(2-hydroxy ethyl)amino]-1-methylbenzimidazol-2-yl]butanoate is disclosed, wherein ethyl-4-

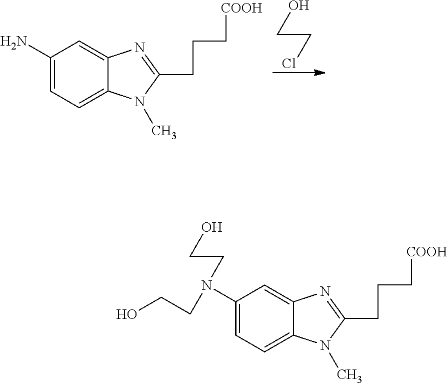

(5-amino-1-methyl-1H-benzo[d]imidazol-2-yl) butanoate is reacted with 2-halo ethanol in the presence of an inorganic base.

Though there are various process disclosures known till date, however, due to one or the other reason either they are particularly not suitable for industrial scale production or there are possible risk in handling highly corrosive or hazardous chemicals or poor yields or isolation difficulties including absence of disclosure of complete process and their parameters. Hence, there still remains a need for industrially viable process for the preparation of bendamustine hydrochloride hydrate.

Regarding the disclosures of polymorphic forms known till date for Bendamustine HCl as well as its monohydrate, WO2009/120386A2 describes solid forms of bendamustine hydrochloride designated as bendamustine hydrochloride Form 1, bendamustine hydrochloride Form 2, bendamustine hydrochloride Form 3, bendamustine hydrochloride Form 4, amorphous bendamustine hydrochloride or a mixture thereof, processes for their preparation along with lyophilized compositions comprising these forms. Further, in this disclosure it is also mentioned that monohydrate of bendamustine hydrochloride has been prepared previously as per W. Ozegowski and D. Krebs. The earlier monohydrate has a reported melting point of 152-156° C. which is similar to that of the observed melting point of Bendamustine hydrochloride Form 2.

In view of the existence of various known polymorphic forms of bendamustine hydrochloride & its monohydrate, there appears to be a need of a convenient process as well as the stable form of bendamustine hydrochloride monohydrate which may be amenable to scale up and provides improved yields & quality.

### SUMMARY OF INVENTION

Different aspects of the present application are summarized herein below individually. In one aspect of the present application, the present invention provides a process of preparation of Bendamustine hydrochloride monohydrate of formula (I)

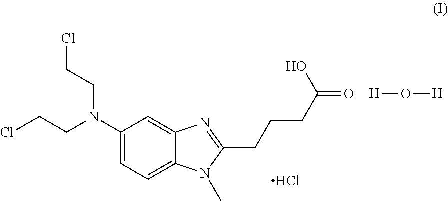

comprising the steps of—
A. reacting 2,4-dinitrochlorobenene (VIII) with aqueous methyl amine solution in alcohol solvent to isolate N-methyl-2,4-dinitroaniline (VII).

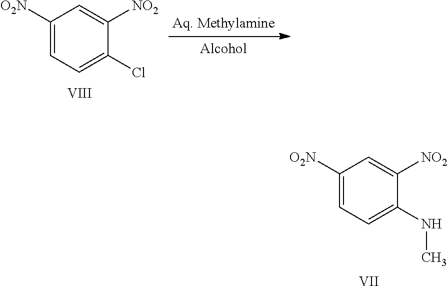

B. Selectively reducing N-methyl-2,4-dinitroaniline (VII) to isolate N¹-methyl-4-Nitrobenzene-1,2-diamine (VI).

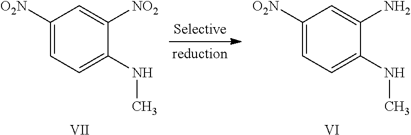

C. Reacting N¹-methyl-4-Nitrobenzene-1,2-diamine (VI) with Dihydro-2H-pyran-2,6(3H)-dione in isopropyl alcohol to isolate Isopropyl 4-(1-methyl-5-nitro-1/H-benzo[d]imidazol-2-yl) butanoate (V)

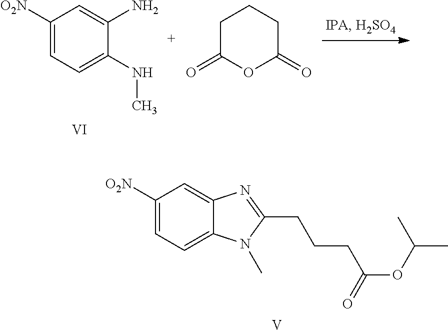

D. Selectively reducing the Isopropyl 4-(1-methyl-5-nitro-1/H-benzo[d]imidazol-2-yl) butanoate (V) with metal reducing agent to isolate Isopropyl 4-(5-amino-1-methyl-1H-benzo[d]imidazol-2-yl) butanoate (IV)

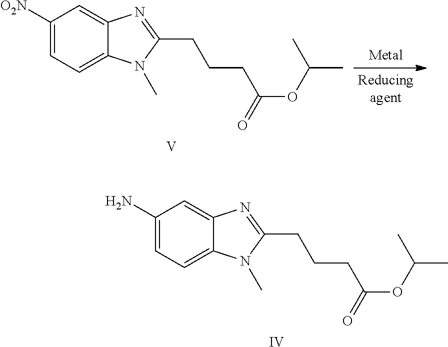

E. Hydroxyethylating the Isopropyl 4-(5-amino-1-methyl-1H-benzo[d]imdazol-2-yl) butanoate (IV) in presence of Diisopropylethylamine (DIPEA) to get Isopropyl 4-(5-bis(2-hydroxyethyl)amino-1-methyl-1H-benzo[d]imidazol-2-yl) butanoate (III).

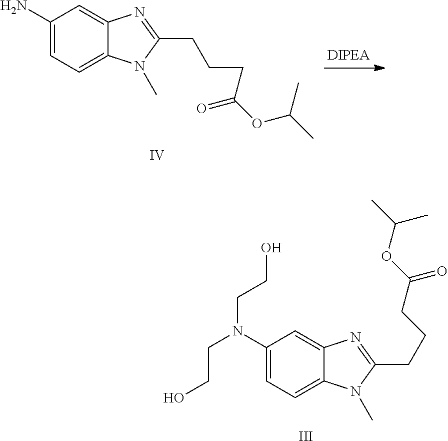

F. Chlorinating the Isopropyl 4-(5-bis(2-hydroxyethyl)amino-1-methyl-1H-benzo[d]imidazol-2-yl) butanoate (IV) and de-esterifying, followed by hydrochlorinating to isolate the Bendamustine HCl monohydrate of formula-I

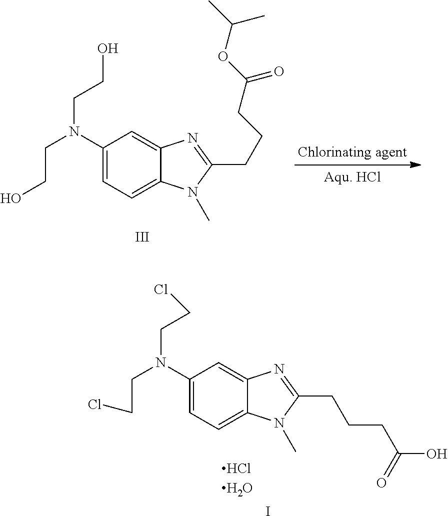

wherein hydrochlorinating in diluted aqueous hydrochloric acid solutions comprising addition of diluted aqueous hydrochloric acid solutions—
   a. the reaction mass is heated upto a temperature ranging between 40 to 65° C.
   b. maintaining the reaction mass at heated temperature till desired acceptable purity profile is attained
   c. cooling the mass to ambient temperature and stirred for time between 1 to 4 hours
   d. isolating the crystalline Bendamustine hydrochloride monohydrate.

In another aspect of the present application, the invention provides a process of purification of Bendamustine hydrochloride or monohydrate comprising the steps of—
a). reacting the crude Bendamustine Hydrochloride anhydrous or its hydrate or mixture thereof obtained from any source with aqueous hydrochloric acid solution
b). heating the contents upto a temperature ranging between 40 to 65° C.
c). maintaining the reaction mass at heated temperature of step e) till desired acceptable purity profile
d). cooling the mass to ambient temperature and stirred for time between 1 to 4 hours.
e). isolating the product as substantially pure crystalline Form-SM
f). optionally repeating the steps b) to step e).

The substantially pure crystalline Form-SM has meaning of purity by HPLC is more than about 98% w/w of polymorphic form designated as crystalline Form-SM.

In yet another aspect of the present application, the invention provides a process of preparation of Bendamustine hydrochloride monohydrate crystalline Form-SM comprising the steps of—
a). reacting the compound of formula IV

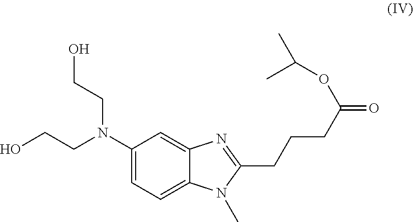 [SMILES: CC(C)OC(=O)CCCc1nc2cc(N(CCO)CCO)ccc2n1C]

with a chlorinating agent in a halogenated hydrocarbon solvent.
b). processing the reaction till completion
c). removing the solvent from the system
d). combining the matter from step c) with aqueous hydrochloric acid solution
e). heating the contents upto a temperature ranging between 40 to 65° C.
f). maintaining the reaction mass at heated temperature of step e) till desired acceptable purity profile
g). cooling the mass to ambient temperature and stirred for time between 1 to 4 hours.
h). isolating the product as crystalline Form-SM
i). optionally repeating the steps d) to step h).

In yet further another aspect of the present application, the invention provides a process of preparation of Bendamustine hydrochloride monohydrate crystalline Form-SM comprising the steps of—
a). reacting the crude Bendamustine or its pharmaceutically acceptable salts and their hydrates thereof obtained from any source with aqueous hydrochloric acid solution
b). heating the contents upto a temperature ranging between 40 to 65° C.
c). maintaining the reaction mass at heated temperature of step e) till desired acceptable purity profile
d). cooling the mass to ambient temperature and stirred for time between 1 to 4 hours.
e). isolating the product as crystalline Form-SM
f). optionally repeating the steps b) to step e).

In a further aspect of the present invention, the application provides a substantially pure Bendamustine hydrochloride monohydrate crystalline Form-SM characterized by X-ray powder diffraction pattern comprising at least 5 characteristic peaks selected from the XRPD 2 theta degrees peaks at 7.42, 10.60, 11.17, 16.43, 17.94, 22.89, 26.33, 28.77, 30.28, 31.92, 40.89±0.1 2θ° and further characterized by DSC thermogram comprising at least two endothermic peaks ranging between—
a. Peak −1—Between 110 to 114° C.
b. Peak −2—Between 125 to 135° C.
c. Peak −3—Between 232 to 238° C.
   wherein substantially pure crystalline Form-SM has meaning of purity by HPLC is more than 98% w/w of polymorphic form designated as crystalline Form-SM.

In a further specific aspect of the present application, the present application provides Bendamustine hydrochloride monohydrate crystalline Form-SM characterized by X-ray powder diffraction pattern comprising at least 7 characteristic peaks selected from the XRPD 2 theta degrees peaks at 7.42, 10.60, 11.17, 16.43, 17.94, 22.89, 26.33, 28.77, 30.28, 31.92, 40.89±0.1 20° and DSC thermogram comprising the endothermic peaks ranging between 110 to 114° C. (Peak −1), 125 to 135° C. (Peak −2) and/or 232 to 238° C. (Peak −3).

### BRIEF DESCRIPTION OF THE DRAWINGS

FIG. 1 is an Illustration of an X-ray powder diffraction (XRPD) pattern of Bendamustine hydrochloride monohydrate, prepared according to Example-1

FIG. 2 is an Illustration of a differential scanning calorimetry (“DSC”) curve of Bendamustine hydrochloride monohydrate, prepared according to Example-1

FIG. 3 is an Illustration of a TGA thermogram of Bendamustine hydrochloride monohydrate, prepared according to Example-1.

### DETAILED DESCRIPTION

As set forth herein, aspects of the present invention provides processes for preparation of Bendamustine Hydrochloride Monohydrate, their purification process, substantially pure product as well as crystalline polymorphic form designated as Form SM.

In one embodiment of the present application, it provides a process of preparation of 1H-benzimidazole-2-butanoic acid, 1-methyl-5-[bis(2-chioroethyl)amino]-, monohydrochloride monohydrate of formula (I) or Bendamustine hydrochloride monohydrate

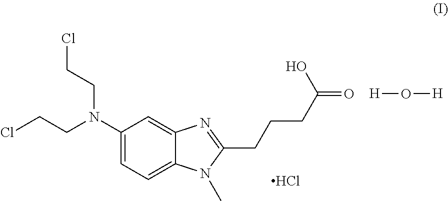

comprising the steps of—
A. Reacting 2,4-dinitrochlorobenene (VIII) with aqueous methyl amine solution in alcohol solvent to isolate N-methyl-2,4-dinitroaniline (VII).

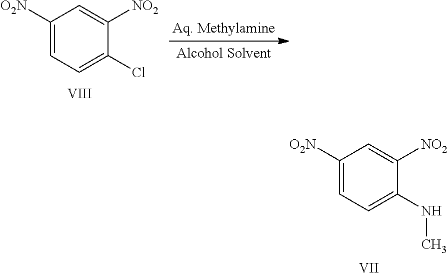

The process of reacting 2,4-dinitrochlorobenene (VIII) with methyl amine comprising the reaction in about equi-molar amounts, however, an excess of molar proportion of methyl amine may be utilized as long as reaction kinetics remain in the control of the desired reaction time, minimal or no impurities formation and any other relevant factor in order to achieve the maximum output of compound of formula-VII. Preferable molar ratio of methyl amine for the reaction may range between about 2 mole to 8 moles of methyl amine per mole of compound of formula VIII. Since the methyl amine is commercially available as aqueous solutions of strengths upto about 40%, accordingly the molar amounts may be calculated. While starting the reaction, the compound of formula VIII is combined with an alcohol solvent, which may be selected from C1 to C4 alcohol. A suitable proportion of alcohol solvent may be desired to get reaction kinetic controlled and preferably about 5 to 10 times the volume of the alcohol solvent may be utilized with respect to total amount of compound of formula-VIII was utilized.

The addition of methyl amine to the alcohol solution of compound of formula-VIII may be carried out at lower temperatures, preferably at temperatures ranging between −5 to +10° C. It may also be preferred to carry out the slow addition of the methyl amine. Time of completion of the reaction depends upon the analytical monitoring confirming the consumption of compound of Formula VIII to a maximum extent, however, without compromising the quality parameters of desired product (Formula VII). In one embodiment, the time consumed for this reaction at room temperature was ranging between about 6-10 hours. The compound of formula-VII may be isolated by conventional methods and optionally may be dried suitably. The conventional methods for isolating the product may include but are not limited to filtering (with or without vacuum), optionally washing with suitable solvent and optional drying.
B. Selectively reducing N-methyl-2,4-dinitroaniline (VII) to isolate N′-methyl-4-Nitrobenzene-1,2-diamine (VI).

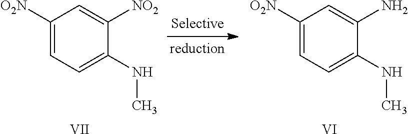

The process of selective reduction of N-methyl-2,4-dinitroaniline (VII) is carried out in an alcohol solvent medium, which may be selected from C1 to C4 alcohol. A suitable proportion of alcohol solvent may be desired to get reaction kinetic controlled and preferably about 6 to 12 times the volume of the alcohol solvent may be utilized with respect to total amount of compound of formula-VII was utilized.

A selective reduction of C-2-nitro group is carried out without affecting the nitro group at C-4 of compound of formula-VII. Conventional reagent like aqueous solution of sodium flakes in alkali or alkali earth metal carbonate may be utilized to carry out this reduction at ambient temperature conditions, followed by raising the reaction mass temperature upto about 50-80° C. Time of completion of the reaction depends upon the analytical monitoring confirming the consumption of compound of Formula VII to a maximum extent, however, without compromising the quality parameters of desired product (Formula VI). In one embodiment, the time consumed for this reaction at room temperature was ranging between about 6-10 hours. The compound of formula-VI may be isolated by conventional methods and optionally may be dried suitably. The conventional methods for isolating the product may include but are not limited to filtering (with or without vacuum), optionally washing with suitable solvent and optional drying.
C. Reacting N¹-methyl-4-Nitrobenzene-1,2-diamine (VI) with Dihydro-2H-pyran-2,6(3H)-dione in isopropyl alcohol to isolate Isopropyl 4-(1-methyl-5-nitro-1/H-benzo[d]imidazol-2-yl) butanoate (V)

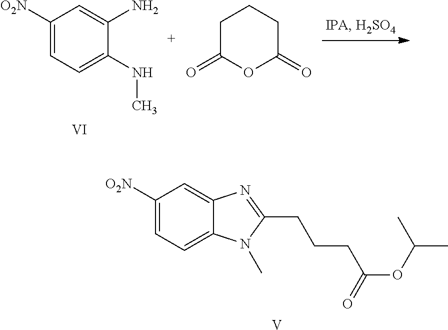

Reaction of N¹-methyl-4-Nitrobenzene-1,2-diamine (VI) with Dihydro-2H-pyran-2,6(3H)-dione in isopropyl alcohol may be carried out at temperatures ranging between about 30 to 50° C. A suitable proportion of isopropyl alcohol solvent may be desired to get reaction kinetic controlled and preferably about 8 to 14 times the volume of the isopropyl alcohol solvent may be utilized with respect to total amount of compound of formula-VI was utilized.

The process of reacting 2,4-dinitrochlorobenene (VI) with Dihydro-2H-pyran-2,6(3H)-dione comprising the reaction in about equi-molar amounts, however, an excess of molar proportion of Dihydro-2H-pyran-2,6(3H)-dione may be utilized as long as reaction kinetics remain in the control of the desired reaction time, minimal or no impurities formation and any other relevant factor in order to achieve the maximum output of compound of formula-V. Preferable molar ratio of Dihydro-2H-pyran-2,6(3H)-dione for the reaction may range between about 1 mole to 3 moles per mole of compound of formula VI.

Time of completion of the reaction depends upon the analytical monitoring confirming the consumption of compound of Formula VI to a maximum extent. In one embodiment, the time consumed for this reaction at temperatures ranging between about 30 to 50° C. was ranging between about 5-10 hours.

Addition of sulphuric acid may be suitably carried out at temperatures ranging between about 30 to 50° C., which may further comprise the temperature elevation upto about 80° C. or preferably reflux temperature of the reaction mass for a suitable time duration.

The compound of formula-V may be isolated by conventional methods and optionally may be dried suitably. The conventional methods for isolating the product may include but are not limited to cooling the reaction mass, wherever required neutralizing, maintaining, filtering (with or without vacuum), optionally washing with suitable solvent and optional drying.
D. Selectively reducing the Isopropyl 4-(1-methyl-5-nitro-1/H-benzo[d]imidazol-2-yl) butanoate (V) with metal reducing agent to isolate Isopropyl 4-(5-amino-1-methyl-1H-benzo[d]imidazol-2-yl) butanoate (IV)

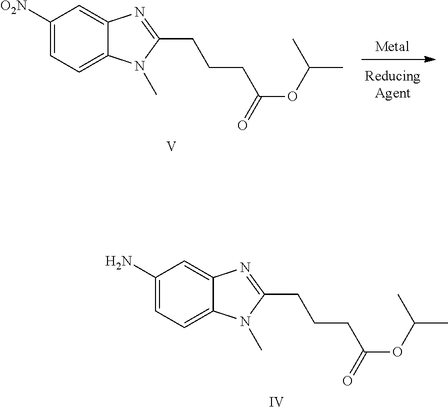

The process of selective reduction of Isopropyl 4-(1-methyl-5-nitro-1/H-benzo[d]imidazol-2-yl) butanoate (V) is carried out in an alcohol solvent medium, which may be selected from C1 to C4 alcohol. A suitable proportion of alcohol solvent may be desired to get reaction kinetic controlled and preferably about 15 to 25 times the volume of the alcohol solvent may be utilized with respect to total amount of compound of formula-V was utilized.

A selective reduction of nitro group is carried out without affecting the isopropyl ester group and the aromatic ring of compound of formula-V. Conventional reagent like metal reducing agents in presence of hydrogen gas may be utilized to carry out this reduction at ambient temperature conditions; however, a hydrogen gas pressure may range between 75 to 105 PSI. The metal reducing agents utilized include Raney Nickel, or similar transition metal group elements. Time of completion of the reaction depends upon the analytical monitoring confirming the consumption of compound of Formula V to a maximum extent, however, without compromising the quality parameters of desired product (Formula IV). In one embodiment, the time consumed for this reaction at room temperature was ranging between about 5-10 hours. The compound of formula-IV may be isolated by conventional methods and optionally may be dried suitably. The conventional methods for isolating the product may include but are not limited to filtering (with or without vacuum), optionally washing with suitable solvent and optional drying.
E. Hydroxyethylating the Isopropyl 4-(5-amino-1-methyl-1H-benzo[d]imidazol-2-yl) butanoate (IV) in presence of Diisopropylethylamine (DIPEA) to get Isopropyl 4-(5-bis(2-hydroxyethyl)amino-1-methyl-1H-benzo[d]imidazol-2-yl) butanoate (III).

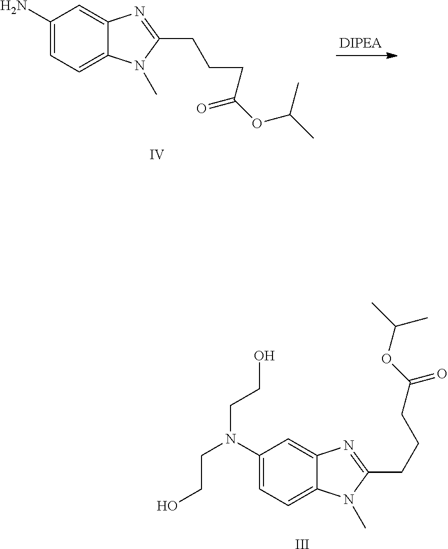

The hydroxyethylation of Isopropyl 4-(5-amino-1-methyl-1H-benzo[d]imidazol-2-yl) butanoate (IV) in presence of Diisopropylethylamine (DIPEA) may be carried out using 2-haloethanol in the aqueous based reaction medium, which may include water or water miscible solvent systems.

The process of hydroxyethylation of Isopropyl 4-(5-amino-1-methyl-1H-benzo[d]imidazol-2-yl) butanoate (IV) with 2-haloethanol comprising the reaction in at least about two molar amounts of 2-haloethanol, however, an excess of molar proportion of 2-haloethanol may be utilized as long as reaction kinetics remain in the control of the desired reaction time, minimal or no impurities formation and any other relevant factor in order to achieve the maximum output of compound of formula-III Preferable molar ratio of 2-haloethanol for the reaction may range between about 2.5 mole to 5.5 moles per mole of compound of formula IV.

Time of completion of the reaction depends upon the analytical monitoring confirming the consumption of compound of Formula IV to a maximum extent. In one embodiment, the time consumed for this reaction at temperatures ranging between about 80 to 95° C. was ranging between about 6-10 hours.

The compound of formula-III may be isolated by conventional methods and optionally may be dried suitably. The conventional methods for isolating the product may include but are not limited to solvent extraction, distillation, filtering (with or without vacuum), optionally washing with suitable solvent and optional drying.
F. Chlorinating the Isopropyl 4-(5-bis(2-hydroxyethyl)amino-1-methyl-1H-benzo[d]imidazol-2-yl) butanoate (III) and isolating the Bendamustine HCl monohydrate of formula-I

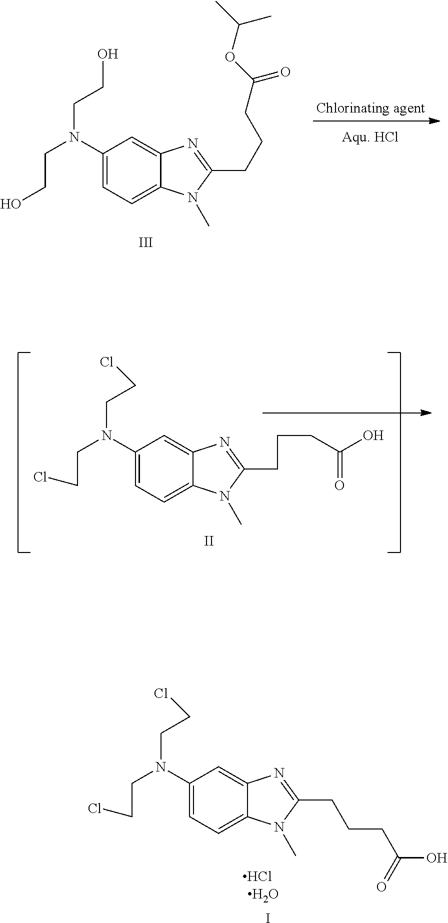

The process of converting the Isopropyl 4-(5-bis(2-hydroxyethyl)amino-1-methyl-1H-benzo[d]imidazol-2-yl) butanoate (III) by chlorinating followed by de-esterification, hydrochlorination and hydration to get Bendamustine HCl monohydrate of formula-I involves the primary reaction of chlorinating agent with compound of formula-III. The chlorination reaction is carried out in inert halohydrocarbon solvent selected from dichloromethane, dichloroethane, trichloromethane, tetrachloromethane or the like.

A suitable proportion of halohydrocarbon solvent may be desired to get reaction kinetics controlled and preferably about 10 to 20 times the volume of the halohydrocarbon solvent may be utilized with respect to total amount of compound of formula-III was utilized. In one of the preferred embodiments, halohydrocarbon solvent was used about 14 times the volume with respect to the total amount of compound of formula-III.

Non-limiting examples of Chlorinating agent that can utilized for the reaction may be selected from thionyl chloride, sulfuryl chloride, phosphorous tri chloride, phosphorous penta chloride

The process of chlorination of Isopropyl 4-(5-bis(2-hydroxyethyl)amino-1-methyl-1H-benzo[d]imidazol-2-yl) butanoate (III) with a chlorinating agent like thionyl chloride comprising the reaction in at least about two molar amounts of thionyl chloride, however, an excess of molar proportion of thionyl chloride may be utilized as long as reaction kinetics remain in the control of the desired reaction time, minimal or no impurities formation and any other relevant factor in order to achieve the maximum output. Preferable molar ratio of thionyl chloride for the reaction may range between about 2.2 mole to 3.0 moles per mole of compound of formula III. In one of the preferred embodiment, about 2.7 moles of thionyl chloride was utilized per mole of compound of formula III.

In view of exothermic nature of the reaction, it may be preferred to carry out the slow or sequential lot wise addition of thionyl chloride. Time of completion of the reaction depends upon the analytical monitoring confirming the consumption of compound of Formula III to a maximum extent. In one embodiment, the time consumed for this reaction at temperatures ranging between about 40 to 70° C. was ranging between about 1-4 hours.

The compound of formula-II may be isolated by conventional methods and optionally may be dried suitably. The conventional methods for isolating the product may include but are not limited to solvent extraction, distillation, filtering (with or without vacuum), optionally washing with suitable solvent and optional drying.

Optionally “insitu” de-esterification, hydrochlorination and hydration may be carried out without isolating the compound of formula II in diluted aqueous hydrochloric acid solutions wherein after addition of diluted aqueous hydrochloric acid solutions—
a. the reaction mass is heated upto a temperature ranging between about 40 to 65° C. Diluted aqueous hydrochloric acid solutions utilized in this step may have dilution ranging between about 4-15% w/w. In one of the preferred embodiment, about 7% w/w aqueous hydrochloric acid solution was utilized. The process of hydrochlorination of Isopropyl 4-(5-bis(2-hydroxyethyl)amino-1-methyl-1H-benzo[d]imidazol-2-yl) butanoate (III) with hydrochloric acid comprising the reaction in atleast about two molar amounts of hydrochloric acid, however, an excess of molar proportion of hydrochloric acid may be utilized as long as reaction kinetics remain in the control of the desired reaction time, minimal or no impurities formation and any other relevant factor in order to achieve the maximum output. Preferable molar ratio of hydrochloric acid for the reaction may range between about 2.1 mole to 3.0 moles per mole of compound of formula III. In one of the preferred embodiment, about 2.7M of hydrochloric acid in the aqueous solution was utilized per mole of compound of formula III.
b. maintaining the reaction mass at heated temperature till desired acceptable purity profile is attained.
c. cooling the mass to ambient temperature and stirred for time between 1 to 4 hours.
d. isolating the crystalline Bendamustine hydrochloride monohydrate.

The step of diluted aqueous hydrochloric acid solution treatment may optionally be repeated, if desired in order to get substantially pure crystalline Bendamustine hydrochloride monohydrate form-SM.

The overall process of the present invention can be summarized in the Scheme-I

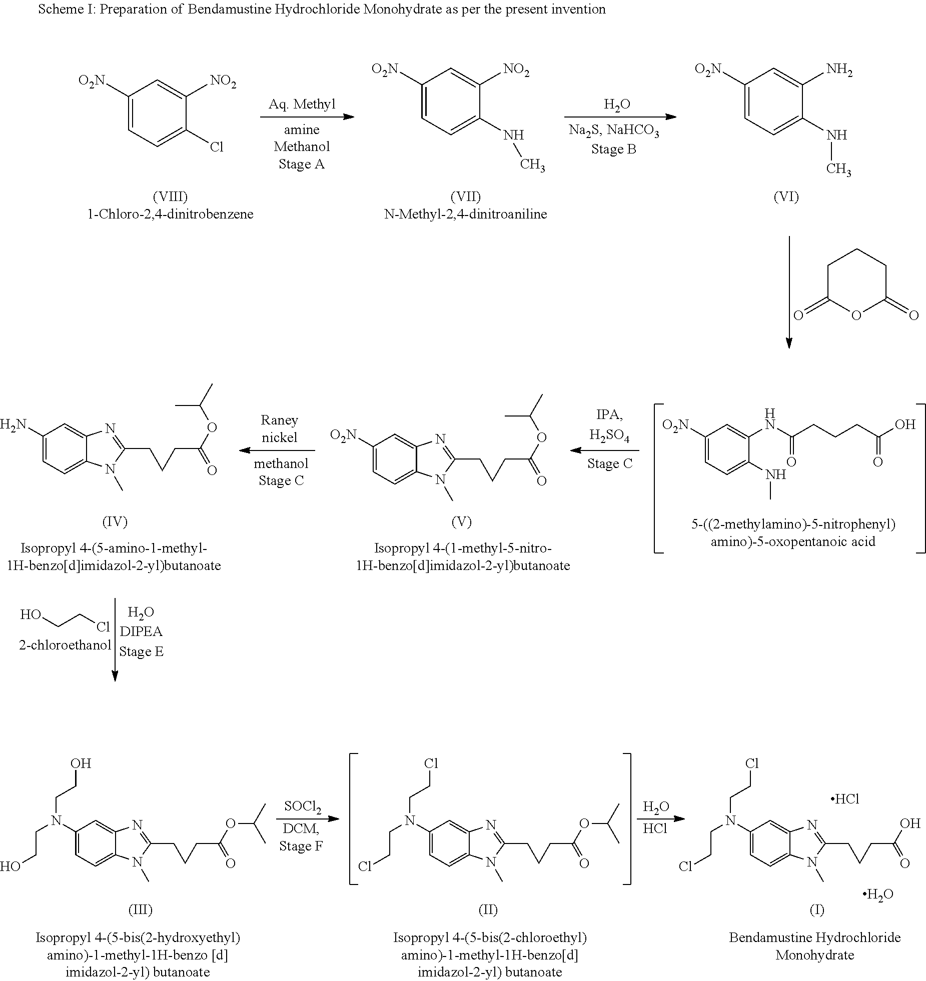

In another embodiment, the present application provides a process of purification of Bendamustine hydrochloride or monohydrate comprising the steps of—
a). reacting the crude Bendamustine Hydrochloride anhydrous or its hydrate or mixture thereof obtained from any source with aqueous hydrochloric acid solution.

Any crude Bendamustine Hydrochloride anhydrous or its hydrate or mixture thereof may be purified by the process of the present invention, which can provide substantially pure crystalline Bendamustine hydrochloride monohydrate form-SM.

Diluted aqueous hydrochloric acid solutions utilized in this step may have dilution ranging between about 5-15% w/w. In one of the preferred embodiment, about 6-7% w/w aqueous hydrochloric acid solution was utilized.

The process of purification of crude Bendamustine Hydrochloride anhydrous or its hydrate with hydrochloric acid comprising combining atleast about two molar amounts of hydrochloric acid, however, an excess of molar proportion of hydrochloric acid may be utilized as long as reaction kinetics remain in the control of the desired reaction time, minimal or no impurities formation and any other relevant factor in order to achieve the maximum output. Preferable molar ratio of hydrochloric acid for the reaction may range between about 1.0 mole to 3.0 moles per mole of crude Bendamustine Hydrochloride anhydrous or its hydrate or mixture thereof. In one of the preferred embodiment, about 2.5 to 2.7M of hydrochloric acid in the aqueous solution was utilized per mole of crude Bendamustine Hydrochloride anhydrous or its hydrate taken earlier.
b). heating the contents upto a temperature ranging between 40 to 65° C.
c). maintaining the reaction mass at heated temperature of step e) till desired acceptable purity profile
d). cooling the mass to ambient temperature and stirred for time between 1 to 4 hours.
e). isolating the product as substantially pure crystalline Form-SM
f). optionally repeating the steps b). to step e).

The substantially pure crystalline Form-SM has meaning of purity by HPLC is more than 98% w/w of polymorphic form designated as crystalline Form-SM.

In yet another embodiment, the present application provides a process of preparation of Bendamustine hydrochloride monohydrate crystalline Form-SM comprising the steps of—
a). reacting the compound of formula IV

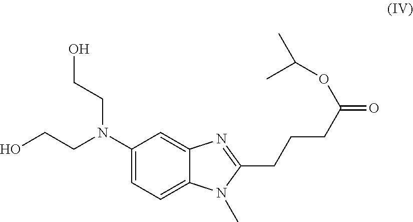 [SMILES: CC(C)OC(=O)CCCc1nc2cc(N(CCO)CCO)ccc2n1C]

with a chlorinating agent in a halogenated hydrocarbon solvent.
b). processing the reaction till completion
c). removing the solvent from the system
d). combining the matter from step c) with aqueous hydrochloric acid solution
e). heating the contents upto a temperature ranging between 40 to 65° C. maintaining the reaction mass at heated temperature of step e) till desired acceptable purity profile
g). cooling the mass to ambient temperature and stirred for time between 1 to 4 hours.
h). isolating the product as crystalline Form-SM
i). optionally repeating the steps d). to step h).

In a further embodiment of the present application, the invention provides a process of preparation of Bendamustine hydrochloride monohydrate crystalline Form-SM comprising the steps of—
a). reacting the crude Bendamustine or its pharmaceutically acceptable salts and their hydrates thereof obtained from any source with aqueous hydrochloric acid solution.
   Any crude Bendamustine or its pharmaceutically acceptable salts and their hydrates thereof may be utilized to convert into Bendamustine hydrochloride monohydrate crystalline Form-SM, by the process of the present invention.
   Aqueous hydrochloric acid solutions utilized in this step may have dilution ranging between about 5-15% w/w. In one of the preferred embodiment, about 6-7% w/w aqueous hydrochloric acid solution was utilized.
   The process of preparation of Bendamustine hydrochloride monohydrate crystalline Form-SM from crude Bendamustine or its pharmaceutically acceptable salts and their hydrates comprise combining with hydrochloric acid upto atleast about one molar amounts of hydrochloric acid, however, an excess of molar proportion of hydrochloric acid may also be utilized as long as reaction kinetics remain in the control of the desired reaction time, minimal or no impurities formation and any other relevant factor in order to achieve the maximum output. Preferable molar ratio of hydrochloric acid for the reaction may range between about 1.0 mole to 3.0 moles per mole of crude Bendamustine Hydrochloride or its pharmaceutically acceptable salts or its hydrate or mixture thereof. In one of the preferred embodiment, about 2.7M of hydrochloric acid in the aqueous solution was utilized per mole of crude Bendamustine or its pharmaceutically acceptable salts or its hydrate taken earlier.
b). heating the contents upto a temperature ranging between 40 to 65° C.
c). maintaining the reaction mass at heated temperature of step b) till desired acceptable purity profile
d). cooling the mass to ambient temperature and stirred for time between 1 to 4 hours.
e). isolating the product as crystalline Form-SM
f). optionally repeating the steps b). to step e).

In yet another embodiment, the present application provides a substantially pure Bendamustine hydrochloride monohydrate crystalline Form-SM characterized by X-ray powder diffraction pattern comprising at least 5 characteristic peaks selected from the XRPD 2 theta degrees peaks at 7.42, 10.60, 11.17, 16.43, 17.94, 22.89, 26.33, 28.77, 30.28, 31.92, 40.89±0.1 20° and further characterized by DSC thermogram comprising at least two endothermic peaks ranging between—
a. Peak −1—Between 110 to 114° C.
b. Peak −2—Between 125 to 135° C.
c. Peak −3—Between 232 to 238° C.
   wherein substantially pure crystalline Form-SM has meaning of purity by HPLC is more than 98% w/w of polymorphic form designated as crystalline Form-SM.

Minor variations in the observed 2 θ° angles values may be expected based on the analyst person, the specific XRPD diffractometer employed and the sample preparation technique. Further possible variations may also be expected for the relative peak intensities, which may be largely affected by the non-uniformity of the particle size of the sample. Hence, identification of the exact crystalline form of a compound should be based primarily on observed 2 theta angles with lesser importance attributed to relative peak intensities. The 2 theta diffraction angles and corresponding d-spacing values account for positions of various peaks in the X-ray powder diffraction pattern. D-spacing values are calculated with observed 2 theta angles and copper Kα wavelength using the Bragg equation well known to those of having skill in the art of XRPD diffractometry science.

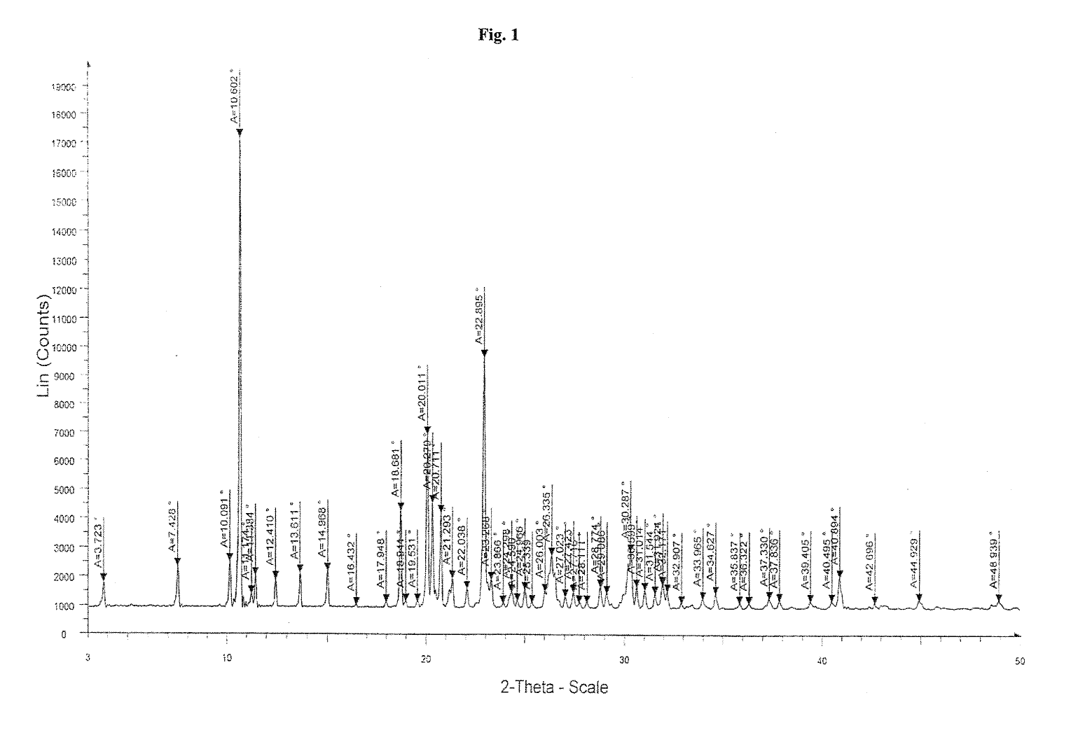

In view of possibility of marginal error in the assigning 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form. For example, one skilled in the art can overlay an X-ray powder diffraction pattern of an unidentified crystalline form of Bendamustine hydrochloride monohydrate over FIG. 1 and readily determine whether the X-ray diffraction pattern of the unidentified form is substantially the same as the X-ray powder diffraction pattern of the crystalline form of this invention. If the X-ray powder diffraction pattern is substantially the same as FIG. 1, the previously unknown crystalline form of Bendamustine hydrochloride monohydrate can be readily and accurately identified as the crystalline Form SM of this invention.

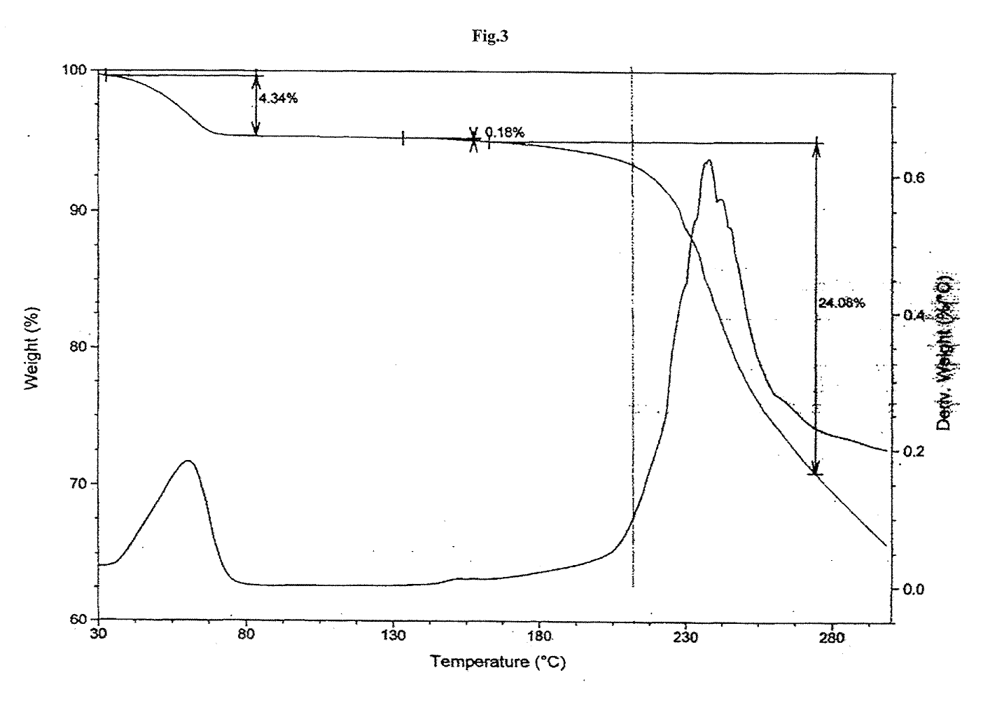

The crystalline Form-SM of Bendamustine hydrochloride is a monohydrate, which may be evident from the FIG. 3 showing the TGA thermogram. A sample of the crystalline Form SM prepared by the inventors had moisture content upto about 4.5% w/w by KF method, which also confirmed the monohydrate nature of the compound. While the invention is not limited to any specific theory, it should be understood however that the crystalline form SM of Bendamustine monohydrochloride monohydrate may contain additional residual or unbound moisture without losing its hydrate character and/or its monohydrate crystalline form-SM characteristics. Nevertheless, one of the skill in the art should be able to determine whether they are same crystalline forms or not, by looking at the overall shape of the X-ray powder diffraction pattern optionally with help of other thermal data like DSC or TGA.

In a particular specific embodiment of the present invention, it provides Bendamustine hydrochloride monohydrate crystalline Form-SM characterized by X-ray powder diffraction pattern comprising at least 7 characteristic peaks selected from the XRPD 2 theta degrees peaks at 7.42, 10.60, 11.17, 16.43, 17.94, 22.89, 26.33, 28.77, 30.28, 31.92, 40.89±0.1 2θ° and DSC thermogram comprising the endothermic peaks ranging between 110 to 114° C. (Peak −1), 125 to 135° C. (Peak −2) and/or 232 to 238° C. (Peak −3).

Crystalline Bendamustine Hydrochloride Monohydrate obtained by the above mentioned process (As per Scheme-I) of the present inventions results in the characteristic polymorphic form XRPD pattern, which is designated as “Form SM”. A polymorphic form may be described by reference to patterns, spectra, or other graphical data as “substantially” shown or depicted in a figure, or by one or more data points. It will be appreciated that patterns, spectra, and other graphical data may be slightly shifted in their positions, relative intensities, or other values due to various factors known to the person skilled in the art. For example, in the crystallographic and powder X-ray diffraction science, shifts in peak positions or the relative intensities of one or more peaks of a pattern can occur because of, the equipment used, protocol of the sample preparation, preferred packing and orientations, the radiation source, operator's minor operational error, method and length of data collection, and the like. However, those of ordinary skill in the art will be able to compare the figures herein with patterns, etc. generated for an unknown form of, in this case, Bendamustine Hydrochloride Monohydrate, and confirms its identity with “Form SM” disclosed herein. The same holds true for other techniques which may be reported herein.

Bendamustine Hydrochloride Monohydrate “Form SM” obtained by the process of present application is characterized by its X-ray powder diffraction (“XRPD”) pattern, differential scanning calorimetry (“DSC”) curve, and thermo gravitational analysis (“TGA”) data.

Substantially pure Bendamustine Hydrochloride Monohydrate “Form SM” can be analysed by HPLC method, using High Perform Liquid chromatograph make Agilent 1200 series equipped with UV Detector operated at 230 nm and Zobraz SB-C18, 250 mm×4.6 mm ID, 5 μm, particle size column. Analyses were performed using the following mobile phase, at flow rate of 1.0 ml/minute column over temperature 30° C.±2° C.

Mobile phase A: Mix 900 ml water containing 0.1% Trifluoroacetic acid and 100 ml Acetonitrile, filter and degas.

Mobile phase B: Mix 500 ml Acetonitrile, 500 ml water containing 0.1% Trifluoroacetic acid, filter and degas.

Elution: Gradient program

TABLE

| Time | Mobile Phase A % | Mobile Phase B % |
|---|---|---|
| 0 | 100 | 0 |
| 3 | 100 | 0 |
| 16 | 50 | 50 |
| 33 | 30 | 70 |
| 35 | 10 | 90 |
| 50 | 10 | 90 |
| 55 | 100 | 0 |
| 60 | 100 | 0 |

In addition, where a reference is made to a figure, it is permissible to, and this document includes and contemplates, the selection of any number of data points illustrated in the figure that uniquely define that crystalline form, within any associated and recited margin of error, for purposes of identification.

All percentages and ratios used herein are by weight of the total composition and all measurements made are at about 25° C. and about normal pressure, unless otherwise designated. All temperatures are in degrees Celsius unless specified otherwise. As used herein, “comprising” (open ended) means the elements recited, or their equivalent in structure or function, plus any other element or elements which are not recited. The terms “having” and “including” are also to be construed as open ended. As used herein, “consisting essentially of” means that the invention may include ingredients in addition to those recited in the claim, but only if the additional ingredients do not materially alter the basic and novel characteristics of the claimed invention. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

The crystalline solid (referred to as ‘Form SM’) exhibits an X-ray powder diffraction pattern substantially as shown in FIG. 1. The prominent and characteristic 2 θ° values for the Form-SM of the present invention, includes 7.42, 10.60, 11.17, 16.43, 17.94, 22.89, 26.33, 28.77, 30.28, 31.92, 40.89±0.1 2θ°.

The crystalline solid ‘Form SM’ described herein may be characterized by X-ray powder diffraction pattern (XRPD), Thermal techniques such as differential scanning calorimetry (DSC) and TGA thermal Analysis. The samples of Bendamustine HCl monohydrate Form-SM were analyzed by XRPD on a Bruker AXS D8 Advance Diffractometer using X-ray source—Cu Kα radiation using the wavelength 1.5418 Å. DSC on a perkin Elmer Pyris 7.0 instrument. Illustrative examples of analytical data for the crystalline solid ‘Form SM’ obtained in the Examples are set forth in the FIGS. 1-3.

In another embodiment, the crystalline “Form SM” of Bendamustine HCl monohydrate obtained by the process of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerine, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising Crystalline Form-SM of Bendamustine HCl monohydrate of the present application include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions of Crystalline Form-SM of Bendamustine HCl monohydrate of the present application may also comprise to include the pharmaceutically acceptable carrier used for the preparation of solid dispersion, wherever utilized in the desired dosage form preparation.

Certain specific aspects and embodiments of the present application will be explained in more detail with reference to the following examples, which are provided by way of illustration only and should not be construed as limiting the scope of the invention in any manner.

### EXAMPLES

### Example 1

### Preparation of Bendamustine Hydrochloride Monohydrate

The process for the preparation of Bendamustine hydrochloride monohydrate comprises the following stages, namely Stages A-F. Individual stages from Stage-A to Stage-F are provided separately herein below—

### Stage A). Preparation of N-methyl-2,4-dinitroaniline (VII)

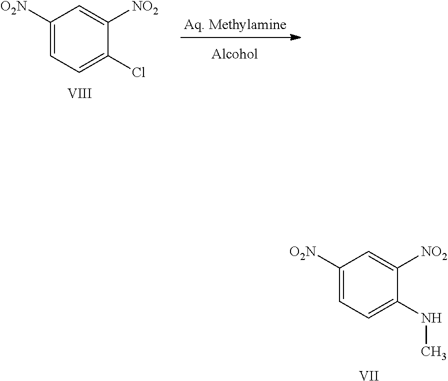

Charged I-Lot of 1800 ml methanol and 250 gm of 2,4-dinitrochlorobenene (VIII) into a round bottom flask at room temperature. Cooled the reaction mass temperature 0-5° C. Charged 426 ml 40% aq. solution of methylamine at the same temperature. Raised the reaction mass temperature to 25-30° C. Stirred the reaction mass for 9-10 hours at 25-30° C. Filtered the solid and washed with II-Lot of 1000 ml methanol. Dried the material under high vacuum in oven for 4-5 hours at 25-30° C. Unloaded the material.

Dry weight: 200-225 gm

### Stage B). Preparation of methyl-4-Nitrobenzene-1,2-diamine (VI)

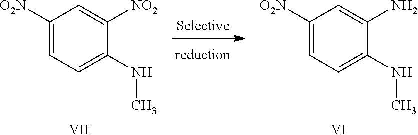

Charged 2000 ml of methanol and 200 gm of stage A product into a round bottom flask. Prepared a solution by the dissolution of sodium sulphide flakes 237.3 gm and sodium bi carbonate 256 gm in lot-I 3000 ml purified water at 25-30° C. and added this solution to the reaction mass at 25-30° C. Raised reaction mass temperature to 70-80° C. (reflux) and stirred for 7-8 hours at 70-80° C. Cooled the reaction mass to 25-30° C. then stirred for 30 minutes at 25-30° C. Filtered the solid and washed with lot-II 1300 ml purified water. Suck it dried for 30 minutes. Dried the material under high vacuum in oven for 4-5 hours at 28-32° C. Unloaded the material.

Dry weight: 133-145.5 gm

### Stage C). Preparation of Isopropyl 4-(1-methyl-5-nitro-1/H-benzo[d]imidazol-2-yl) butanoate (V)

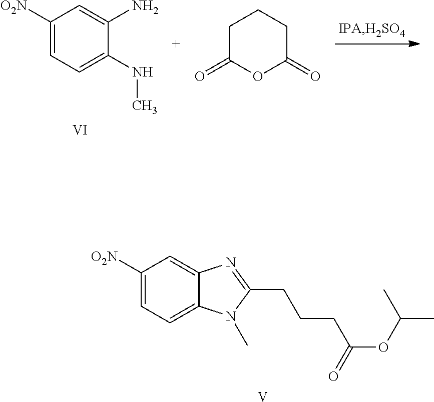

Charged 1750 ml Isopropyl alcohol and 140 gm stage B product into round bottom flask. Charged 143.4 gm Dihydro-2H-pyran-2,6(3H)-dione into the round bottom flask. Raised the reaction mass temperature to 35-40° C. and stirred for 5-6 hours at the same temperature. Added 44 ml conc. sulphuric acid into the at 35-40° C. Raised the reaction mass temperature to 80-85° C. (reflux) and stirred for 8-9 hours. Then cooled the reaction mass to 25-30° C. Prepared a solution of potassium carbonate dissolved in 7400 ml lot I purified water in another round bottom flask at 25-30° C. Slowly added the reaction mass to the prepared solution at 25-30° C. Stirred the reaction mass for 2-3 hours at 25-30° C. Filtered the solid and washed with 896 ml lot II purified water. Suck it dried for 30 minutes. Dried the material under high vacuum in oven for 4-5 hours at 25-30° C. Unloaded the material.

Dry weight: 140-168 gm

### Stage D). Preparation of Isopropyl 4-(5-amino-1-methyl-1H-benzo[d]imidazol-2-yl) butanoate (IV)

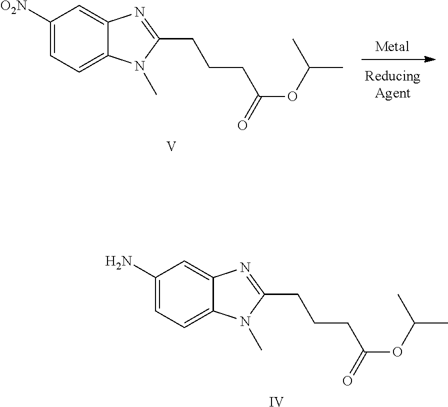

Charged 150 gm of Stage-C product into auto clave (Pressure vessel). Charged 3000 ml lot-I methanol into auto clave. Charged 42 gm raney nickel into an auto clave. Applied the 90-100 PSI of hydrogen gas. Maintained the reaction mass at 25-30° C. and 90-100 PSI of hydrogen gas for 7-8 hours. Checked the TLC for absence of Stage-C product. Unload the reaction mass from autoclave and washed the autoclave with 750 ml lot-II Methanol. Filtered the reaction mixture over cellite bed and washed with 750 ml lot-III methanol. Distillout the filtrate up to 1 to 1.5 volumes at below 50° C. under vacuum. Added 4500 ml lot-I purified water and kept under stirring. Stirred reaction mass for 1-2 hours at 25-30° C. Filtered the solid and washed with 960 ml lot-II purified water. Suck dried for 30 minutes. Dried the material for 3-4 hrs at 45-50° C. Unloaded the material.

Dry weight: 60-90 gm

### Stage E). Preparation of Isopropyl 4-(5-bis(2-hydroxyethyl)amino-1-methyl-1H-benzo[d]imidazol-2-yl) butanoate (III)

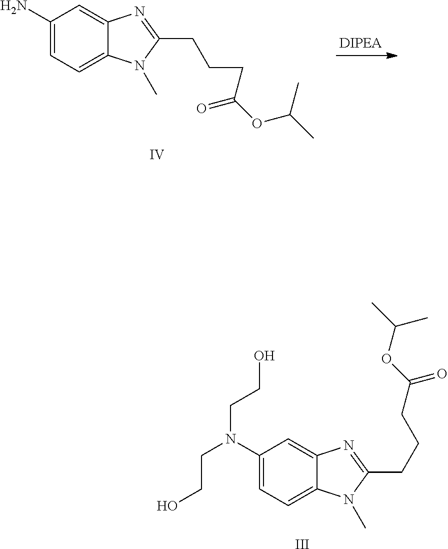

Charged DM water into round bottom flask at 25-30° C. Added 70 gm product of Stage-D into round bottom flask at 25-30° C. Charged 70 ml 2-chloroethanol into it at 25-30° C. Charged 140 gm diisopropyl ethyl amine (DIPEA) into it at 25-30° C. Raised the reaction mass temperature to 90-95° C. Maintained the reaction mass at 90-95° C. for 8-9 hours. If reaction completes, cooled the reaction mass to 25-30° C. Added 350 ml lot-I dichloromethane into the reaction mass at 25-30° C. and stirred for 10 minutes. Separated the two layers. Extracted the aqueous layer with 280 ml of lot-II and 280 ml of lot-III dichloromethane. Combined organic layer washed with lot-I(280 ml) and lot-II(280 ml) saturated sodium chloride solution. Distill out the organic layer upto one volume at below 45° C. under vacuum. Added 280 ml lot-I ethyl acetate and concentrated upto one volume at below 45° C. under vacuum. Added 140 ml lot-II ethyl acetate at 45° C. and cooled to 25-30° C. Collected the reaction mass at 0-5° C. Maintained the reaction mass at 0-5° C. for 2-3 hours. Filtered the solid and washed with cold 56 ml lot-III ethyl acetate (0-5° C.). Suck it dried for 15 minutes. Dried the compound at 45-50° C. in oven with high vacuum for 4-5 hours.

Dry Weight: 43.0 gm

### Stage F). Preparation of Isopropyl 4-(5-bis(2-hydroxyethyl)amino-1-methyl-1H-benzo[d]imidazol-2-yl) butanoate hydrochloride monohydrate (I)

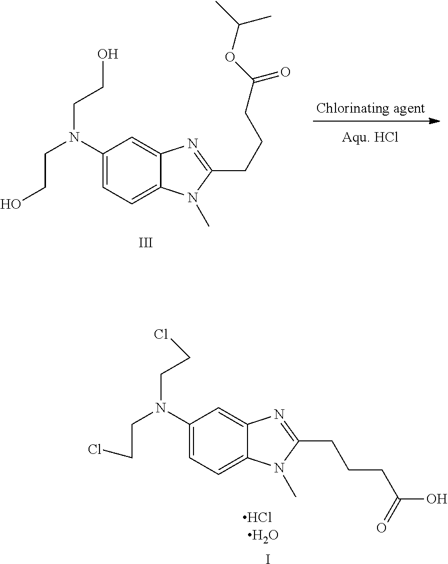

Charge 600 ml lot I dichloromethane in round bottom flask at 25-30° C. and then charge stage E product. Slowly add 24 ml Thionyl chloride drop wise below 30° C. (exothermic reaction). Raise the temperature to 55-60° C. and maintained for 2 hrs. Cooled the reaction mass to 25-30° C. Added 400 ml of dichloromethane. Added 200 ml lot I purified water and separated the layer. Combined the organic layer and wash it with 200 ml saturated sodium bicarbonate solution. Combined the organic layer and wash it with 200 ml saturated sodium chloride solution. Settle and separated the layer. Combined the organic layer and dry it over sodium sulphate. Distill out the reaction mass under vacuum at 40° C. upto one volume. Then added 370 ml N-heptane into the reaction mass and stirred for 2 hrs at room temperature. Filter the reaction mass and washed it with 10 ml N-heptane and suck dried for 1 hr. Taken the in situ mass in round bottom flask and added 40 ml dilute hydrochloric acid into the reaction mass. Slowly heat the reaction mass to 55-60° C. Maintained for 7-8 hrs (checking and confirming the impurity profile). Cool the reaction mass to room temperature and stir for 3-4 hrs. Filter the reaction mass and wash it with 2 ml chilled water.

Again Charge crude Bendamustine Hydrochloride monohydrate isolated in the wet stage in round bottom flask containing 136 ml purified water and 40 ml hydrochloric acid (˜30%) at ambient temperature. Stir the solution for about 30 minutes at 25-30° C. Raise the temperature at 55-60° C. and maintained for about 6-8 hrs (This time may be more, however, depending upon achieving equilibration to impurity profile compliance). Cool the reaction mass upto 25° C. followed by stir for 2 hrs. Filter the reaction mass and wash it with 10 ml purified water (cold).

Yield—16.40 gm

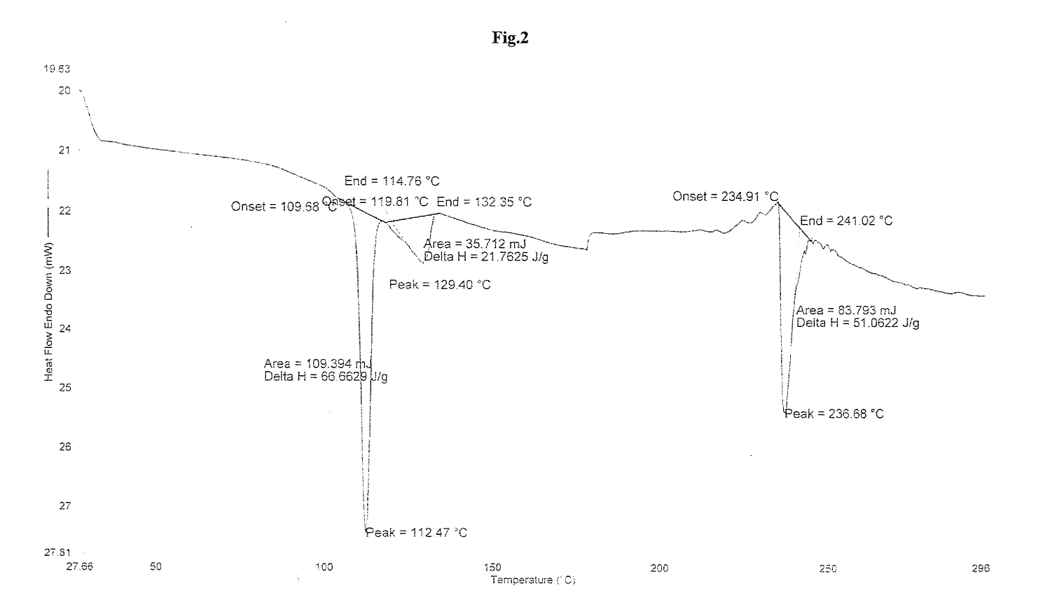

XRPD as per FIG. 1; DSC as per FIG. 2 and TGA as per FIG. 3

### Example 2

### Preparation of Crystalline Bendamustine Hydrochloride Monohydrate (Form-SM)

Charge 270 ml DM water at ambient temperature followed by slow addition of 80 ml hydrochloric acid (˜35%) in round bottom flask. Add ˜35-40 gm crude Bendamustine Hydrochloride Monohydrate obtained from any source and stirred for about 20-25 minutes. Raise the temperature upto about 55-60° C. and maintained for about 6-8 hrs. (This time may be more, however, depending upon achieving equilibration to impurity profile compliance). Cool the reaction mass upto 25-30° C. and stir for about 2 hrs at 25-30° C. Filter the reaction mass followed by washing with 15-20 ml purified cold water and isolating the product after drying.

Yield—30 gm

### Example 3

### Preparation of Substantially Pure Crystalline Bendamustine Hydrochloride Monohydrate (Form-SM)

Charge 20 ml DM water at ambient temperature followed by slow addition of 80 ml hydrochloric acid (˜35%) in round bottom flask. Add 40 gm crude Bendamustine Hydrochloride Monohydrate obtained from any source and stirred for about 20-25 minutes. Raise the temperature upto about 50-55° C. and maintained for about 4-6 hrs. (This time may be more, however, depending upon achieving equilibration to impurity profile compliance). Cool the reaction mass upto 25-30° C. and stir for about 2 hrs at 25-30° C. Filter the reaction mass followed by washing with 15-20 ml purified cold water and isolating the product after drying.

Yield—28-28.5 gm

HPLC purity—99.5 to 99.7%

We claim:

1. Bendamustine hydrochloride monohydrate crystalline Form-SM characterized by X-ray powder diffraction pattern as depicted in FIG. 1 consisting peaks selected from the XRPD 2 theta degrees peaks at 7.42, 10.60, 11.17, 16.43, 17.94, 22.89, 26.33, 28.77, 30.28, 31.92, and 40.89±0.1 2θ° having a purity of greater than 99.5% (by HPLC).

2. Bendamustine hydrochloride monohydrate crystalline Form-SM according to claim 1 which is further characterized by X-ray powder diffraction pattern as depicted in FIG. 1 consisting peaks selected from the XRPD 2 theta degrees peaks at 7.42, 10.60, 11.17, 16.43, 17.94, 22.89, 26.33, 28.77, 30.28, 31.92, and 40.89±0.1 2θ° and DSC thermogram (obtained with hermetically sealed aluminum pan without hole), comprising of endothermic peaks ranging between 110 to 114° C. (Peak −1), 125 to 135° C. (Peak −2) and/or 232 to 238° C. (Peak −3) as depicted in FIG. 2 having a purity of greater than 99.5% (by HPLC).

3. Bendamustine hydrochloride monohydrate crystalline Form-SM according to claim 1, which is further characterized by DSC isothermal pattern consisting endothermic peaks ranging between 110 to 114° C. (Peak −1), 125 to 135° C. (Peak −2) and/or 232 to 238° C. (Peak −3) as depicted in FIG. 2 having a purity of greater than 99.5% (by HPLC).

4. Bendamustine hydrochloride monohydrate crystalline Form-SM characterized by X-ray powder diffraction pattern consisting of peaks selected from the XRPD 2 theta degrees peaks at 7.42, 10.60, 11.17, 16.43, 17.94, 22.89, 26.33, 28.77, 30.28, 31.92 and 40.89±0.1 2θ° having a purity of greater than 99.5% (by HPLC) prepared by a process comprising the steps of—
a) reacting the crude Bendamustine or its pharmaceutically acceptable salts and their hydrates thereof obtained from any source with diluted aqueous hydrochloric acid solution;
b) heating the contents up to a temperature ranging between 40 to 65° C.;
c) maintaining the reaction mass at heated temperature of step b) till desired acceptable purity profile is attained;
d) cooling the mass to ambient temperature and stirring for time between 1 to 4 hours;
e) isolating the product as substantially pure crystalline Form-SM;
f) optionally repeating the steps b) to e).

5. A process for preparing the Bendamustine hydrochloride monohydrate crystalline Form-SM having a purity of greater than 99.5% (by HPLC) according to claim 4, wherein pharmaceutically acceptable salts of step a) is hydrochloride salt.

6. A process for preparing the Bendamustine hydrochloride monohydrate crystalline Form-SM having a purity of greater than 99.5% (by HPLC) according to claim 4, wherein diluted aqueous hydrochloric acid solution utilized in step a) is having dilution ranging between 5-15% w/w.

7. A process for preparing the Bendamustine hydrochloride monohydrate crystalline Form-SM having a purity of greater than 99.5% (by HPLC) according to claim 4, wherein diluted aqueous hydrochloric acid solution utilized in step a) is having molar ratio of hydrochloric acid ranging between 1-3.0 moles per mole of Bendamustine or its pharmaceutically acceptable salts.

8. A process of preparation of crystalline Bendamustine hydrochloride monohydrate crystalline Form-SM having a purity of greater than 99.5% (by HPLC) of formula (I), 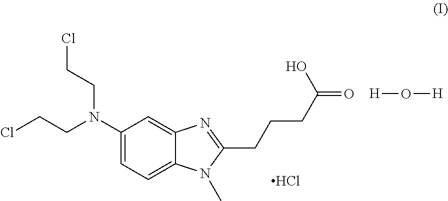
comprising the steps of—
a) reacting 2,4-dinitrochlorobenzene (VIII) with aqueous methyl amine solution in alcohol solvent to isolate N-methyl-2,4-dinitroaniline (VII); 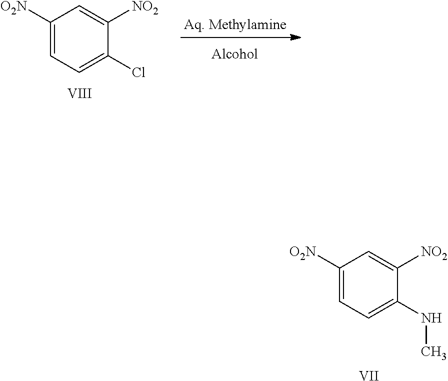
b) selectively reducing N-methyl-2,4-dinitroaniline (VII) to isolate N¹-methyl-4-Nitrobenzene-1,2-diamine (VI); 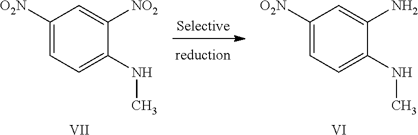
c) reacting N¹-methyl-4-Nitrobenzene-1,2-diamine (VI) with Dihydro-2H-pyran-2,6(3H)-dione in isopropyl alcohol to isolate Isopropyl 4-(1-methyl-5-nitro-1/H-benzo[d]imidazol-2-yl) butanoate (V); 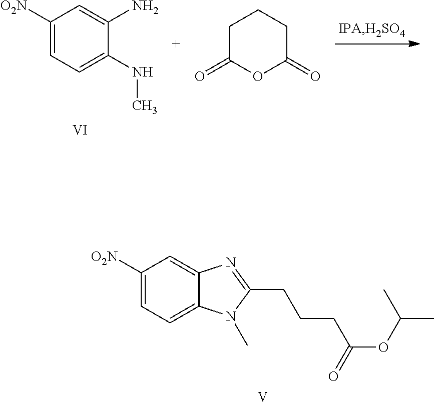
d) selectively reducing the Isopropyl 4-(1-methyl-5-nitro-1/H-benzo[d]imidazol-2-yl) butanoate (V) with metal reducing agent to isolate Isopropyl 4-(5-amino-1-methyl-1H-benzo[d]imidazol-2-yl) butanoate (IV); 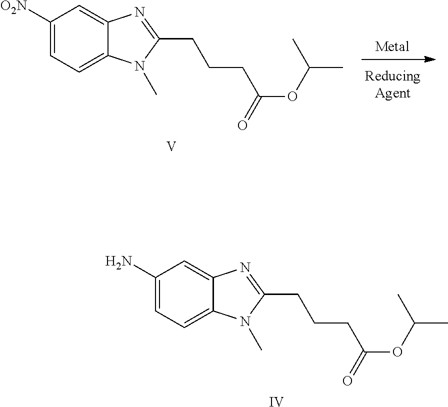
e) hydroxyethylating the Isopropyl 4-(5-amino-1-methyl-1H-benzo[d]imidazol-2-yl) butanoate (IV) in presence of Diisopropylethylamine (DIPEA) and haloethanol to get Isopropyl 4-(5-bis(2-hydroxyethyl)amino-1-methyl-1H-benzo[d]imidazol-2-yl)butanoate (III); and 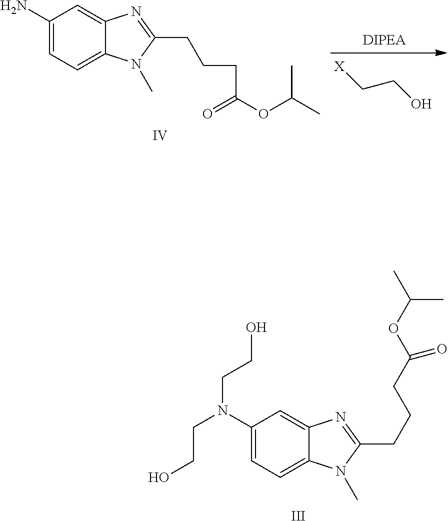
f) chlorinating the Isopropyl 4-(5-bis(2-hydroxyethyl)amino-1-methyl-1H-benzo[d]imidazol-2-yl)butanoate (III) with a chlorinating agent and de-esterifying, followed by hydrochlorinating to isolate the Bendamustine HCl monohydrate of formula-I; 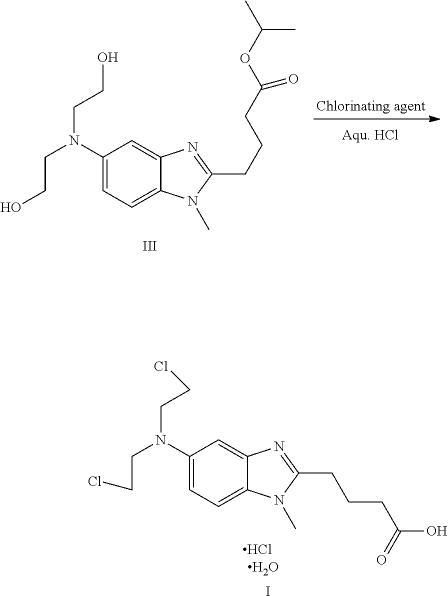
wherein hydrochlorinating in diluted aqueous hydrochloric acid solutions comprising addition of diluted aqueous hydrochloric acid solutions—
i. the reaction mass is heated up to a temperature ranging between 40 to 65° C.;
ii. maintaining the reaction mass at heated temperature till desired acceptable purity profile is attained;
iii. cooling the mass to ambient temperature and stirring for time between 1 to 4 hours; and
iv. isolating the crystalline Bendamustine hydrochloride monohydrate in Form-SM.

9. A process of preparation of Bendamustine hydrochloride monohydrate crystalline Form-SM having a purity of greater than 99.5% (by HPLC) according to claim-8, wherein metal reducing agent used in step d) is Raney Nickel or similar transition metals.

10. A process of preparation of Bendamustine hydrochloride monohydrate crystalline Form-SM according to claim 8 comprising the steps of,
a) reacting the crude Bendamustine or its pharmaceutically acceptable salts and their hydrates thereof obtained from any source with aqueous hydrochloric acid solution;
b) heating the contents upto a temperature ranging between 40 to 65° C.;
c) maintaining the reaction mass at heated temperature of step b) till desired acceptable purity profile is attained;
d) cooling the mass to ambient temperature and stirring for time between 1 to 4 hours:
e) isolating the product as crystalline Form-SM;
f) optionally repeating the steps b) to step e).
